(19) United States
(12) Patent Application Publication
(10) Pub. No.: US 2007/0098826 A1
(43) Pub. Date: May 3, 2007

(54) Use of pinitol or chiroinositol for protecting the liver

(75) Inventors: Yong Chul Shin, Jinju-si (KR); Yeong Joong Jeon, Seoul (KR); Jong Jin Kim, Jinju-si (KR)

Correspondence Address: SUGHRUE MION, PLLC, 2100 PENNSYLVANIA AVENUE, N.W., SUITE 800, WASHINGTON, DC, 20037, US

(73) Assignee: AMICOGEN, INC., Kyungsangnam-do (KR)

(21) Appl. No.: 10/550,272

(22) PCT Filed: Mar. 26, 2004
(86) PCT No.: PCT/KR04/00689

§ 371 Date: Sep. 23, 2005

(30) Foreign Application Priority Data
Mar. 26, 2003 (KR) 10-2003-0019018

(51) Int. Cl. A61K 31/075; A01N 31/14; A61K 36/48; A61K 36/13; A01N 65/00

(52) U.S. Cl. 424/757; 514/715; 424/770

(57) ABSTRACT

Disclosed in this invention is a use of pinitol or chiroinositol for protecting the liver.

### FIELD OF THE INVENTION

The present invention relates to a use of pinitol (C₇H₁₄O₆, MW 194.18), chiroinositol (C₆H₁₂O₆, MW 180.16) or an extract of a plant containing pinitol or chiroinositol for protecting the liver.

### BACKGROUND OF THE INVENTION

The number of the population afflicted by various types of liver diseases have recently been on the increase due to dietary changes, stress, excessive intake of alcohol, and/or hepatotoxic substances. Liver cirrhoisis, in particular, which is caused by alcohol, drug, chemicals, metabolic diseases such as viral hepatitis and biliary disease, or autoimmunity diseases, suppress the liver function by lowering both the hepatic blood flow and metabolic enzyme activity and by changes in proteins in the blood and bile flow.

The hepatic function deteriorates and may develop into hepatitis, hepatocirrhosis or hepatic cancer as a result of excessive intake of alcohol or foods having a high lipid content, or infection by hepatitis B or C virus. In particular, the excessive intake of fat-containing foods and alcohol causes fatty liver leading to elevated levels of serum GOT (glutamate-oxaloacetate transaminase), GPT (glutamate-pyruvate transaminase) and γ-GTP (γ-glutamyl transpeptidase).

Oxidative stress also plays an important role in the attack by alcoholic liver-related diseases, non-alcoholic fatty liver-related diseases and viral liver-related diseases (Arteel G E, Gastroenterology, 2003, 124: 778-90; Loguercio C and Federico A. Free Radic. Biol, Med., 2003, 1; 34(1): 1-10; Mehta K et al., Nutr. Rev., 2002, 60(9): 289-93; Gebhardt R. Planta Med., 2002, 68(4): 289-96; Adachi M et al., Free Radic. Biol. Med., 2002, 15; 32(6): 487-91; Parola M et al., J. Hepatol., 2001, 35(2): 297-306). Superoxide dismutase (SOD), an anti-oxidation enzyme, participates in the treating or preventing liver-related diseases by way of mitigating the oxidative stress. It has also been reported that glutathione plays an important role as a non-enzymatic anti-oxidant in the protection of cells from the demage by radicals and also in the synthesis of proteins or DNA, material transportation and enzyme reactions.

Pinitol, which is metabolized into chiroinositol in the body, has been reported to be effective in treating or preventing fatness, hyperlipidemia and hypertension (U.S. Pat. No. 5,550,166). However, pharmacological activity of pinitol or chiroinositol in preventing or treating liver-related diseases has never been explored.

### SUMMARY OF THE INVENTION

Accordingly, it is an object of the present invention to provide a pharmacologically active substance for preventing and treating liver-related diseases by protecting the liver.

### DETAILED DESCRIPTION OF THE INVENTION

In accordance with one aspect of the present invention, there is provided a use of pinitol or chiroinochitol for protecting the liver in a mammal

In accordance with one aspect of the present invention, there is provided a use of an extract of a plant containing pinitol or chiroinochitol for protecting the liver in a mammal

The plant which may be used in the present invention is inclusive of soybean, pine, Hovenia dulcis Thunb, Acanthopanax senticosus, carob and the like, and preferably soybean and carob.

The extract of a plant containing pinitol or chiroinositol of the present invention can be prepared using such a solvent as water or an organic solvent, e.g., a lower alcohol, acetone, chloroform, methylenechloride, ether, ethylacetate, hexane and a mixture thereof. Examples of the lower alcohol are methanol, ethanol, propanol and butanol, preferably ethanol.

The plant used in the extraction procedure of the present invention may be of a dried powder form. Specifically, a water extract of a plant can be prepared by adding 5 to 15 fold volume of water, preferably 10-fold volume of water to a dried plant powder, extracting for 1 to 24 hours, preferably 2 to 5 hours at 10 to 80° C., preferably 30 to 50° C., and then filtering. Alternately, 1 to 15-fold volume, preferably 10-fold volume of an organic solvent may be used to extract a plant powder at room temperature, to obtain an organic solvent extract. The above extraction procedure may be repeated two more times as needed. Also, after the filtration, a powder form of the extract can be prepared by removing the solvent under a reduced pressure.

In order to prevent and treat liver-related diseases, or to protect liver, pinitol, chiroinositol or an extract of a plant containing pinitol or chiroinositol can be administered to a mammal in the form of a composition containing, e.g., a pharmaceutical composition, a food composition or a beverage composition.

The content of pinitol or chiroinositol in the pharmaceutical composition of the present invention may range form 10 to 100 wt %, preferably 5 to 50 wt % based on the total weight of the composition, and the amount of the plant extract containing pinitol or chiroinositol in the pharmaceutical composition of the present invention may range form 1 to 50 wt %, preferably 5 to 30 wt % based on the total weight of the composition.

The pharmaceutical composition of the present invention can effectively protect the liver by way of reducing the levels of GOT, GPT and γ GTP in the blood and promoting the superoxide dismutase (SOD) activity and by way of increasing the glutathione level in the liver.

In spite of such potent efficacies, the inventive pharmaceutical composition containing pinitol, chiroinositol, or an extract of a plant containing pinitol or chiroinositol shows little toxicity or mitogenicity in animal tests and exerts no adverse effects on the liver function.

A pharmaceutical formulation may be prepared in accordance with any of the conventional procedures. In preparing the formulation, the active ingredient is preferably admixed or diluted with a carrier, or enclosed within a carrier, sachet or other container. When the carrier serves as a diluent, it may be a solid, semi-solid or liquid material acting as a vehicle, excipient or medium for the active ingredient. Thus, the formulations may be in the form of a tablet, pill, powder, sachet, elixir, suspension, emulsion, solution, syrup, aerosol, soft and hard gelatin capsule, sterile injectable solution, sterile packaged powder and the like.

Examples of suitable carriers, excipients, and diluents are lactose, dextrose, sucrose, sorbitol, mannitol, calcium silicate, cellulose, methyl cellulose, microcrystalline cellulose, polyvinylpyrrolidone, water, methylhydroxybenzoates, propylhydroxybenzoates, talc, magnesium stearate and mineral oil. The formulations may additionally include fillers, anti-agglutinating agents, lubricating agents, wetting agents, flavoring agents, emulsifiers, preservatives and the like. The compositions of the invention may be formulated so as to provide quick, sustained or delayed release of the active ingredient after their administration to a mammal by employing any of the procedures well known in the art.

The pharmaceutical composition of the present invention can be administered via various routes including oral, transdermal, subcutaneous, intravenous and intramuscular introduction. In case of human, a typical daily dose of pinitol or chiroinositol may range from about 0.1 to 100 mg/kg body weight, preferably 1 to 50 mg/kg body weight, and can be administered in a single dose or in divided doses. However, it should be understood that the amount of the active ingredient actually administered ought to be determined in light of various relevant factors including the condition to be treated, the chosen route of administration, the age, sex and body weight of the individual patient, and the severity of the patient's symptom; and, therefore, the above dose should not be intended to limit the scope of the invention in any way.

The present invention also provides a method for preventing or treating liver-related diseases in mammals, which comprises administering thereto an effective amount of pinitol or chiroinositol or the extract of plant containing pinitol or chiroinositol.

Moreover, pinitol, chiroinositol, or the extract of plant containing pinitol or chiroinositol can be incorporated in foods or beverages, as an additive or a dietary supplement, for the purpose of protecting liver. In this case, the content of pinitol or chiroinositol in a food or beverage may range from 0.1 to 50 wt %, preferably 1 to 10 wt % based on the total weight of the food, and 0.01 to 10 g, preferably 0.1 to 1 g of per 100 ml of the beverage.

The health care beverage composition of the present invention may contain other components, e.g., deodorants and natural carbohydrates as in conventional beverages. As the deodorant, a natural deodorant such as taumatin, Stevia extract, e.g., levaudioside A, glycyrrhizin and the like, or a synthetic deodorant such as saccharin and aspartam may be used. Examples of such natural carbohydrates are monosaccharides such as glucose and fructose; disaccharides such as maltose and sucrose; conventional polysaccharides such as dextrin and cyclodextrin; and sugar alcohols such as xylitol, sorbitol and erythritol. The amount of the above-described natural carbohydrate is generally in the range of about 1 to 20 g, preferably 5 to 12 g based on 100 ml of beverage.

Other components that may be added to the inventive food or beverage composition are various nutrients, vitamins, minerals, synthetic flavoring agents, coloring agents, pectic acid and its salt, alginic acid and its salt, organic acids, protective colloidal adhesives, pH controlling agents, stabilizers, preservatives, glycerin, alcohol, carbonizing agents used in carbonated beverage. The amount of the above-described additives is generally in the range of about 0 to 20 weight portions based on 100 weight portions of the composition.

Moreover, the foods containing pinitol, chiroinositol or the extract of plant containing pinitol or chiroinositol and the additional herbal extracts to develop health supplementary food, may include various foods, various beverages, various gums, vitamin complexes.

The following examples are intended to further illustrate the present invention without limiting its scope.

### EXAMPLE 1

### Preparation and Analysis of Plant Extract Containing Pinitol

Soybean, pine needle, Hovenia dulcis Thunb, Acanthopanax senticosus were each dried and pulverized at room temperature and 10 g of the dried powder was extracted with 100 ml of distilled water at 25° C. for 6 hours.

The pinitol content of each extract was measured by High Performance Liquid Chromatography using Dionex Carbopak MA-1 column (eluent: 10 mM NaOH) and the result is shown in Table I.

TABLE I

| Plant | Pinitol content (g/kg) |
|---|---|
| Soybean | 4.4 |
| Pine needle | 6.7 |
| Hovenia dulcis Thunb | 4.0 |
| Acanthopanax senticosus | 4.8 |

### EXAMPLE 2

### Toxicity of Orally Administered Pinitol

6 week-old, specific pathogen-free Sprague-Dawley female rats (15 heads), each weighing about 130 to 147 g, and male rats (15 heads), each weighing about 110 to 123 g, were bred under the condition of 23±3° C., 55±15% relative humidity and 12 L/12 D photoperiod. Fodder (Harlan, U.S.A.) and water were sterilized and fed to the rats. The rats were acclimated for 1 week before the administration of pinitol.

Pinitol was dissolved in physiological saline and the solution was orally administered to each rat in an amount of 5,000 mg/kg of rat body weight. The solution was administered once and the rats were observed for 14 days for signs of adverse effects or death according to the following schedule: every hour for 6 hours after the administration and, every day thereafter. The weight changes of the rats were recorded at day 1, 3, 7 and 14 to examine the effect of pinitol. Further, on day 14, the rats were sacrificed and the internal organs were visually examined.

All the rats were alive at day 14 and pinitol showed no toxicity at a dose of 5,000 mg/kg. The autopsy revealed that the rats did not develop any pathological abnormality, and no weight loss was observed during the 14 day test period. Accordingly, it was concluded that pinitol is not toxic when orally administered to an animal.

### EXAMPLE 3

### Protective Activity for the Liver Damaged by Carbon Tetrachloride

Sprague-Dawley rats (80 heads), each weighing about 180 to 200 g, were bred under the condition of temperature 23±3° C., 55±15% relative humidity and 12 L/12 D photoperiod. Fodder (Harlan, U.S.A.) and water were sterilized and fed to the rats.

The rats were divided into 8 groups and carbon tetrachloride was injected subcutaneously into the rats except the rats of the normal group in an amount of 0.5 ml/kg at 1^{st} and 5^{th} day. On day 2, pinitol or chiroinositol dissolved in 10 ml of water was orally administered to the rats of the experimental groups in an amount of 5-20 mg/kg of rat body weight. The normal and control groups were treated with 10 ml of distilled water instead of pinitol. On day 8, GOT and GPT concentrations in the blood sample taken from the orbital vein of each rat was measured by using blood analyzer (Vitros DT-60, Johnson & Johnson) and the result is shown in Table II. The degree of inhibition (%) was then calculated in accordance with the following equation:

$$Degree of Inhibition ( \% ) = 100 \times [ 1 - \frac{Experimental group - Normal group}{Control group - Normal group} ]$$

TABLE II

| | GOT | | GPT | |
|---|---|---|---|---|
| | | Inhibition | | Inhibition |
| | IU/L | (%) | IU/L | (%) |
| Normal group | 65 ± 8 | | 30 ± 7 | |
| Control group | 522 ± 48 | | 259 ± 22 | |
| Experimental group | 298 ± 28 | 49.7 | 134 ± 7 | 54.6 |
| 1 (Pinitol 5 mg/kg) | | | | |
| Experimental group | 188 ± 22 | 73.4 | 92 ± 5 | 72.9 |
| 2 (Pinitol 10 mg/kg) | | | | |
| Experimental group | 192 ± 17 | 72.6 | 75 ± 4 | 80.3 |
| 3 (Pinitol 20 mg/kg) | | | | |
| Experimental group | 321 ± 35 | 44.0 | 156 ± 12 | 45.0 |
| 4 (chiroinositol 5 | | | | |
| mg/kg) | | | | |
| Experimental group | 205 ± 28 | 69.4 | 80 ± 9 | 78.2 |
| 5 (chiroinositol 10 | | | | |
| mg/kg) | | | | |
| Experimental group | 174 ± 26 | 76.1 | 88 ± 7 | 74.7 |
| 6 (chiroinositol 20 | | | | |
| mg/kg) | | | | |

As can be seen from Table II, the control group rats showed markedly higher GOT and GPT concentrations than those of the normal group. The GOT and GPT concentrations of the pinitol-fed rats group were lower than those of the control group by 49.7˜73.45% and 54.6˜80.3%, respectively, and the chiroinositol-fed rats group, by 44.0˜76.1% and 45.0˜78.2%, respectively. This result demonstrates that pinitol and chiroinositol have distinct liver-protecting activity.

### EXAMPLE 4

### Determination of SOD Activity in Liver

6 week-old, Sprague-Dawley rats (70 heads) were bred under the condition of 23±3° C., 55±15% relative humidity, 12 L/12 D (a.m. 8-p.m. 8) photoperiod and 150-300 Lux illumination. Fodder (Harlan, U.S.A.) and water were sterilized and fed to the rats.

The rats were divided into 7 groups and pinitol dissolved in 10 ml of water was orally administered to the rats of the experimental groups in an amount of 200-1000 mg/kg of rat body weight everyday for 4 days. The comparative group rats were treated with Silymarin (Sigma Chemical Co.) in an amount of 200 mg/kg, the normal and control groups were each treated with 10 ml of distilled water. On day 4, a mixture of carbon tetrachloride and soybean oil (1:1 (v/v)) was injected intraperitoneally into the rats except the rats of the normal group in an amount of 0.5 ml/kg, 90 minutes after the administration of pinitol. 24 hours thereafter, SOD activity of the liver taken out from each rat was measured according to the method of Neoot et al., (Methods Enzymol., 186: 209-219 (1993)) and tested by student t-test. The result is shown in Table III.

TABLE III

| | SOD activity (U/g Liver) | Relative Value (%) |
|---|---|---|
| Normal group | 209 ± 51 | 90 |
| Control group | 233 ± 29 | 100 |
| Comparative group | 191 ± 25 | 82 |
| Experimental group 1 | 217 ± 74 | 93 |
| (pinitol 200 mg/kg) | | |
| Experimental group 2 | 289 ± 45 | 124 |
| (Pinitol 300 mg/kg) | | |
| Experimental group 3 | 335 ± 52 | 144 |
| (Pinitol 500 mg/kg) | | |
| Experimental group 4 | 315 ± 38 | 135 |
| (Pinitol 1000 mg/kg) | | |

As can be seen from Table III, SOD activities of the rats of the comparative group and experimental group 1 were lower than that of the control group by 18% and 7%, respectively, while those of experimental groups 2, 3 and 4 were higher by 24%, 44% and 35% than that of the control group. These results demonstrate that the administration of 300 mg/kg of pinitol raises SOD activity in the liver (p<0.05).

### EXAMPLE 5

### Measurement of Content of Glutathione in Liver

In order to induce diabetes in rats, streptozotocin (Sigma Chemical Co., USA) dissolved in 0.01M citrate buffer solution (pH 4.5) was injected into the abdominal cavity of 8 week-old Sprague-Dawley rats in an amount of 45 mg/kg of rat body weight. After 3 days, the blood glucose value of a venous blood sample taken out from the tail of each rat was measured and the rats having more than 251±7 mg/dl of blood glucose value deemed to the diabetic rats. The diabetic rats were divided into 3 groups, each of 12-15 rats, and pinitol or chiroinositol dissolved in 10 ml of water was orally administered to the rats of two groups (experimental groups 1 and 2) in an amount of 10 mg/kg of rat body weight everyday for 4 days. Rats of the normal group and control group (diabetic group) were each treated with 10 ml of distilled water. After fasting during 18 hours, the rats were anesthetized with ethyl ether, sacrificed, the liver was taken and washed with cold physiological saline. The glutathione content of the homogenized liver solution was measured according to the method of Ellman (Arch. Biochem. Biophy., 70-77 (1959)) and the result is shown in Table IV.

TABLE IV

| | Glutathione content | |
|---|---|---|
| | (μ mole/g) | Relative value |
| Normal group | 17.09 ± 0.77 | 100 |
| Control group | 6.51 ± 1.50 | 38.1 |
| (Diabetic group) | | |
| Experimental group 1 | 16.38 ± 2.43 | 95.8 |
| (pinitol group) | | |
| Experimental group 1 | 14.52 ± 0.57 | 85.0 |
| (chiroinositol group) | | |

As can be seen from Table IV, the glutathione contents for the rats of the control group (diabetic group) was markedly lower than that of the normal group (38%), while those of the experimental group 1 (pinitol group) and experimental group 2 (chiroinositol group) were only slightly lower as compared to the normal group, exhibiting high values of 96% and 85%, respectively.

### EXAMPLE 6

### Protective Activity for Liver

Pinitol was orally administered to adults (the average age: 51.8 years, man 7 and female 8) showing serum GOT or γ-GPT level higher than 50 IU/L at a daily dose of 600 mg for 2 months. Serum GOT, GPT and γ-GTP levels were determined before and after the administration. The results are shown in Table V

TABLE V

| | Before | After | Degree of |
|---|---|---|---|
| | administration | administration | decrease (%) |
| GOT(IU/L) | 57.4 ± 10.6 | 31.4 ± 3.0 | 45.3 |
| GPT(IU/L) | 89.6 ± 22.3 | 43.2 ± 8.1 | 51.8 |
| [symbol] −GTP(IU/L) | 140.2 ± 31.1 | 82.6 ± 17.5 | 41.1 |

As can be see from Table V, serum GOT, GPT and γ-GTP levels decreased by 45.3%, 51.8% and 41.1%, respectively, after the administration.

### FORMULATION EXAMPLES

The composition of the present invention can be used in preparing a pharmaceutical formulation by admixing the active ingredients with pharmaceutical excipients in various pharmaceutical forms according to any one of the conventional methods, as exemplified below without limiting the scope of the present invention.

<Formulation Example 1> Preparation of Powder

| | | |
|---|---|---|
| Pinitol or Chiroinositol | 600 | mg |
| Lactose | 1,400 | mg |

The above ingredients were mixed thoroughly and then, filled and sealed in a sealed package to obtain a powder preparation.

<Formulation Example 2> Preparation of Tablet

| | | |
|---|---|---|
| Pinitol or Chiroinositol | 200 | mg |
| Corn Starch | 50 | mg |
| Lactose | 50 | mg |
| Steric Acid Magnesium | 2 | mg |

The above ingredients were mixed thoroughly and tabletted according to a conventional method to obtain a tablet preparation.

<Formulation Example 3> Preparation of Capsule

| | | |
|---|---|---|
| Pinitol or Chiroinositol | 200 | mg |
| Corn Starch | 100 | mg |
| Lactose | 100 | mg |
| Steric Acid Magnesium | 2 | mg |

The above ingredients were mixed thoroughly and filled in a gelatin capsule according to a conventional method to obtain a capsule preparation.

<Formulation Example 4> Preparation of Injection Solution

| | |
|---|---|
| Pinitol or Chiroinositol | 200 mg |
| Distilled water for injection | q.s. |
| pH adjuster | q.s. |

The above ingredients were dissolved in distilled water for injection, and adjusted to pH approximately 7.5. The resulting solution was filled in 2 ml of ample with distilled water for injection and sterilized according to a conventional method to obtain an injection preparation.

<Preparation of Health Care Beverage>

1-10 wt % of pinitol or chiroinositol, 5-10 wt % of sugar, 0.05-0.3 wt % of citric acid, 0.005-0.02 wt % of caramel and 0.1-1 wt % of vitamin C were mixed and distilled water was added thereto to obtain a syrup. The syrup thus obtained was sterilized at 85-98° C. for 20-180 seconds, mixed with 4-fold volume of cool water, and 0.5 to 0.82% of carbonic acid gas was added thereto, to obtain a carbonated beverage containing pinitol or chiroinositol.

Also, pinitol or chiroinositol was homogeneously mixed with liquid fructose (0.5%), oligosaccharides (2%), sugar (2%), saline (0.5%) and water (75%) and instantaneously sterilized to obtain a health beverage.

While the invention has been described with respect to the above specific embodiments, it should be recognized that various modifications and changes may be made to the invention by those skilled in the art which also fall within the scope of the invention as defined by the appended claims.

What is claimed is:

1. A use of pinitol or chiroinositol for protecting the liver in a mammal.

2. The use of claim 1, wherein pinitol or chiroinositol enhances superoxide dismutase (SOD) activity.

3. The use of claim 1, wherein pinitol or chiroinositol increases the glutathione level in the liver.

4. The use of claim 1, wherein the mammal is human.

5. The use of claim 1, wherein pinitol or chiroinositol is administered to the mammal in the form of a composition containing same, said composition being selected from the group consisting of a pharmaceutical composition, a food composition and a beverage composition.

6. A use of an extract of a plant containing pinitol or chiroinositol for protecting the liver in a mammal.

7. The use of claim 6, wherein the plant extract containing pinitol or chiroinositol enhances superoxide dismutase (SOD) activity.

8. The use of claim 6, wherein the plant extract containing increases the glutathione level in the liver.

9. The use of claim 6, wherein the mammal is human.

10. The use of claim 6, wherein the plant is selected from the group consisted of soybean, pine, Hovenia dulcis Thunb, Acanthopanax senticosus and carob.

11. The use of claim 6, wherein the plant extract is a water extract or an organic solvent extract.

12. The use of claim 11, wherein the plant extract is prepared by adding 5 to 15-fold volume of water to a plant powder; extracting at 10 to 80° C. for 1 to 24 hours; and filtering the extract thus obtained.

13. The use of claim 6, wherein the plant extract containing pinitol or chiroinositol is administered to the mammal in the form of a composition containing same, said composition being selected from the group consisting of: a pharmaceutical composition, a food composition and a beverage composition.

14. A method for preventing or treating a liver disease in a mammal, which comprises administering an effective amount of pinitol, chiroinositol or an extract of a plant containing pinitol or chiroinositol thereto.

15. The method of claim 14, wherein the effective amount of pinitol or chiroinositol is 0.1 to 100 mg/kg body weight/day.
